(19) United States
(12) Patent Application Publication
(10) Pub. No.: US 2015/0157863 A1
(43) Pub. Date: Jun. 11, 2015

(54) METHOD AND APPARATUS FOR THERAPY ADJUSTMENT IN RESPONSE TO INDUCED CARDIAC CONDUCTION CHANGES

(71) Applicant: Cardiac Pacemakers, Inc., St. Paul, MN (US)

(72) Inventors: Jeffrey E. Stahmann, Ramsey, MN (US); Stephen B. Ruble, Lino Lakes, MN (US); David J. Ternes, Roseville, MN (US)

(21) Appl. No.: 14/559,009

(22) Filed: Dec. 3, 2014

Related U.S. Application Data
(60) Provisional application No. 61/912,341, filed on Dec. 5, 2013.

(51) Int. Cl. A61N 1/36; A61N 1/365

(52) U.S. Cl. CPC A61N 1/36139; A61N 1/365

(57) ABSTRACT

An implantable system delivers a plurality of electrical stimulation therapies to a patient and controls the delivery such that a physiologic change induced by at least one of the therapies is detected and used to adjust one or more of the therapies. In various embodiments, the implantable system delivers a cardiac rhythm management (CRM) therapy such as a pacing therapy and a neural stimulation (NS) therapy such as an autonomic modulation therapy (AMT). In various embodiments, the physiologic change includes a change in a cardiac conduction interval that may be detected within a detection window following delivery of the pacing or NS therapy.

### CLAIM OF PRIORITY

This application claims the benefit of priority under 35 U.S.C. §119(e) of U.S. Provisional Patent Application Ser. No. 61/912,341, filed on Dec. 5, 2013, which is herein incorporated by reference in its entirety.

### TECHNICAL FIELD

This document relates generally to medical devices and particularly to a system that delivers multiple electrical stimulation therapies and adjusts one or more therapies in response to a therapy-induced physiologic change such as a change in cardiac conduction.

### BACKGROUND

Different types of therapies can be delivered simultaneously, or near simultaneously, to treat the same condition or to treat different conditions. For example, a patient with cardiovascular disorders may benefit from both neural stimulation (NS) therapy and cardiac rhythm management (CRM) therapy.

While CRM therapy such as cardiac pacing therapy is applied to treat cardiac disorders, some types of NS therapy such as autonomic modulation therapy (AMT) are also known to modulate cardiovascular functions. Some NS therapies can alter cardiac contractility and excitability. Direct electrical stimulation of parasympathetic nerves can activate the baroreflex, inducing a reduction of sympathetic nerve activity and reducing blood pressure by decreasing vascular resistance. Sympathetic inhibition, as well as parasympathetic activation, have been associated with reduced arrhythmia vulnerability following a myocardial infarction, presumably by increasing collateral perfusion of the acutely ischemic myocardium and decreasing myocardial damage. Parasympathetic activation may alter refractory period of the exited tissue, thereby preventing arrhythmias from developing. Modulation of the sympathetic and parasympathetic nervous system with neural stimulation has been shown to have positive clinical benefits, such as protecting the myocardium from further remodeling and predisposition to fatal arrhythmias following a myocardial infarction.

Because the CRM and NS therapies may affect the same physiologic condition, and that physiologic condition may be used to control either or both of the CRM and NS therapies, there is a need for coordinating the therapies in the context of therapy-induced changes in the physiologic condition.

### SUMMARY

An implantable system delivers a plurality of electrical stimulation therapies to a patient and controls the delivery such that a physiologic change induced by at least one of the therapies is detected and used to adjust one or more of the therapies. In various embodiments, the implantable system delivers a cardiac rhythm management (CRM) therapy such as a pacing therapy and a neural stimulation (NS) therapy such as an autonomic modulation therapy (AMT). In various embodiments, the physiologic change includes a change in a cardiac conduction interval that may be detected within a detection window following delivery of the pacing or NS therapy.

In one embodiment, a system for delivering a plurality of electrical stimulation therapies to a patient's body includes a stimulation circuit. The stimulation circuit is configured to deliver a first type electrical stimulation to the body and includes a pulse output circuit and a control circuit. The pulse output circuit is configured to deliver stimulation pulses. The control circuit is configured to control the delivery of the stimulation pulses using a plurality of stimulation parameters and includes a signal receiver, a physiologic change detector, and a parameter adjuster. The signal receiver is configured to receive a signal indicative of delivery of a second type electrical stimulation. The physiologic change detector is configured to start a detection window in response to receipt of the signal and detect a physiologic change during the detection window. The parameter adjuster is configured to adjust at least one parameter of the plurality of stimulation parameters in response to a detection of the physiologic change.

In one embodiment, a method for delivering a plurality of electrical stimulation therapies to a patient's body is provided. Stimulation pulses of a first type electrical stimulation are delivered. The delivery of the stimulation pulses is controlled using a plurality of stimulation parameters of the first type electrical stimulation. The controlling includes receiving a signal indicative of delivery of a second type electrical stimulation, starting a detection window in response to the signal being received, detecting a physiologic change during the detection window, and adjusting at least one parameter of the plurality of stimulation parameters in response to the physiologic change being detected.

This Summary is an overview of some of the teachings of the present application and not intended to be an exclusive or exhaustive treatment of the present subject matter. Further details about the present subject matter are found in the detailed description and appended claims. Other aspects of the invention will be apparent to persons skilled in the art upon reading and understanding the following detailed description and viewing the drawings that form a part thereof. The scope of the present invention is defined by the appended claims and their legal equivalents.

### BRIEF DESCRIPTION OF THE DRAWINGS

The drawings illustrate generally, by way of example, various embodiments discussed in the present document. The drawings are for illustrative purposes only and may not be to scale.

FIG. 1 is a block diagram illustrating an embodiment of a system including two stimulation circuits.

FIG. 2 is a block diagram illustrating an embodiment of a system including a pacing circuit and a neural stimulation (NS) circuit.

FIG. 3 is a block diagram illustrating an embodiment of a circuit for the system of FIG. 2.

FIG. 4 is an illustration of an embodiment of an implantable system for modulating cardiovascular functions.

FIG. 5 is an illustration of another embodiment of an implantable system for modulating cardiovascular functions.

FIG. 6 is a flow chart illustrating an embodiment of a method for adjusting a pacing therapy when an autonomic modulation therapy (AMT) is delivered.

FIG. 7 is a flow chart illustrating an embodiment of another method for adjusting a pacing therapy when an AMT is delivered.

FIG. 8 is a flow chart illustrating an embodiment of another method for adjusting a pacing therapy when an AMT is delivered.

FIG. 9 is a flow chart illustrating an embodiment of another method for adjusting a pacing therapy when an AMT is delivered.

FIG. 10 is a flow chart illustrating an embodiment of a method for adjusting an AMT therapy.

FIG. 11 is a flow chart illustrating an embodiment of another method for adjusting an AMT therapy.

FIG. 12 is a flow chart illustrating another embodiment of a method for adjusting an AMT therapy when a pacing therapy is delivered.

FIG. 13 is a flow chart illustrating an embodiment of a method for adjusting an AMT therapy when a cardiac event is detected.

FIG. 14 is a flow chart illustrating an embodiment of a method for adjusting an AMT therapy when a premature ventricular contraction (PVC) is detected.

### DETAILED DESCRIPTION

In the following detailed description, reference is made to the accompanying drawings which form a part hereof, and in which is shown by way of illustration specific embodiments in which the invention may be practiced. These embodiments are described in sufficient detail to enable those skilled in the art to practice the invention, and it is to be understood that the embodiments may be combined, or that other embodiments may be utilized and that structural, logical and electrical changes may be made without departing from the spirit and scope of the present invention. The following detailed description provides examples, and the scope of the present invention is defined by the appended claims and their legal equivalents.

It should be noted that references to “an”, “one”, or “various” embodiments in this disclosure are not necessarily to the same embodiment, and such references contemplate more than one embodiment.

This document discusses a system and method for delivering electrical stimulation therapies to a patient and controlling the delivery such that a physiologic change in the patient induced by at least one of the therapies is detected and used to adjust one or more of the therapies. Examples of the electrical stimulation therapies include cardiac rhythm management (CRM) therapies such as pacing therapy and neural stimulation (NS) therapies such as autonomic modulation therapy (AMT). Such therapies may be applied to treat the same cardiovascular disorder or related disorders in the patient. Effects of one therapy delivered to the patient may affect certain physiologic parameters in the patient, which may in turn affect the efficacy of another therapy, especially when the therapies are concurrently or simultaneously delivered. For example, it is reported that AMT can alter cardiac conduction. See, e.g., Wallick et al., “Sympathetic and periodic vagal influences on antegrade and retrograde conduction through the canine atrioventricular node”, Circulation, 76:830-836 (1986). It is also known that atrial pacing, ventricular pacing, biventricular pacing, and premature ventricular contraction can alter cardiac conduction. The present system and method adjust cardiac pacing therapy and/or AMT to account for the effect of cardiac pacing and/or AMT on one or more physiologic conditions such as the cardiac condition. In various embodiments, cardiac pacing therapy and AMT are temporally coordinated to provide for better control of cardiovascular function and improved efficacy for both therapies.

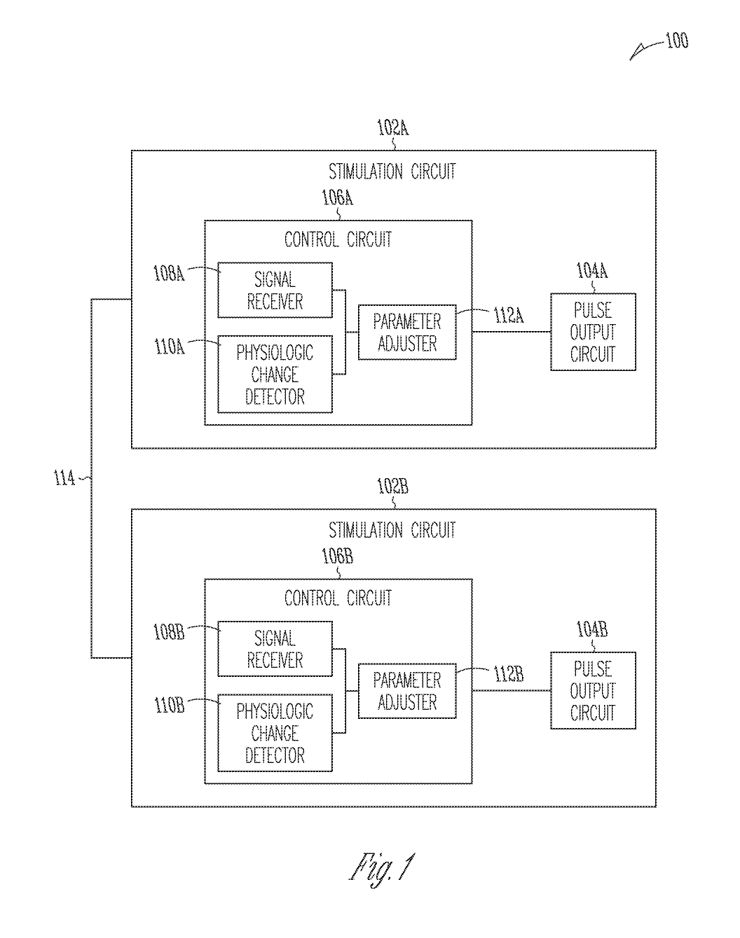

FIG. 1 is a block diagram illustrating an embodiment of a system 100 including stimulation circuits 102A and 102B. In various embodiments, system 100 is configured for delivering two or more electrical stimulation therapies to a patient's body. The stimulation therapies each apply at least one type of electrical stimulation.

Stimulation circuit 102A delivers at least a first type electrical stimulation to the patient's body and includes a pulse output circuit 104A and a control circuit 106A. Pulse output circuit 104A delivers first stimulation pulses (i.e., pulses of the first type electrical stimulation) or first non-pulse energy (i.e., the first type electrical stimulation delivered in a continuous form of energy). Control circuit 106A controls the delivery of the first stimulation pulses using a plurality of first stimulation parameters. In the illustrated embodiment, control circuit 106A includes a signal receiver 108A, a physiologic change detector 110A, and a parameter adjuster 112A. In various embodiments, control circuit 106A can include parameter adjuster 112A and either or both of signal receiver 108A and physiologic change detector 110A.

In one embodiment, signal receiver 108A receives a first signal indicative of delivery of a second type electrical stimulation, such as from stimulation circuit 102B. Parameter adjuster 112A adjusts at least one parameter of the plurality of first stimulation parameters in response to a receipt of the first signal. In another embodiment, physiologic change detector 110A detects a first type physiologic change indicative of delivery of the second type electrical stimulation. Parameter adjuster 112A adjusts at least one parameter of the plurality of first stimulation parameters in response to a detection of the first type physiologic change. In another embodiment, signal receiver 108A receives the first signal indicative of delivery of the second type electrical stimulation, such as from stimulation circuit 102B. Physiologic change detector 110A starts a first detection window in response to the receipt of the first signal, and detects the first type physiologic change during the first detection window. Parameter adjuster 112A adjusts at least one parameter of the plurality of first stimulation parameters in response to a detection of the first type physiologic change during the first detection window.

In various embodiments, to detect the first type physiologic change, physiologic change detector 110A receives a physiologic signal, produces a physiologic parameter using the physiologic signal, and compares the physiologic parameter to a threshold. Parameter adjuster 112A adjusts at least one parameter of the plurality of first stimulation parameters in response to the physiologic parameter exceeding the threshold. Examples of the first type physiologic change include changes in cardiac conductions as an expected response to the delivery of the second type electrical stimulation. More specific examples of the first type physiologic change include changes in cardiac conduction intervals, such as a change in atrioventricular (AV) interval (time interval between intrinsic atrial and ventricular events in an electrogram), PR interval (time interval between P and R waves in an electrocardiogram), or a change to the width of the QRS complex.

Stimulation circuit 102B delivers at least the second type electrical stimulation to the patient's body and includes a pulse output circuit 104B and a control circuit 106B. Pulse output circuit 104B delivers second stimulation pulses (i.e., pulses of the second type electrical stimulation) a second non-pulse energy (i.e., the second type electrical stimulation delivered in a continuous form of energy). Control circuit 106B controls the delivery of the second stimulation pulses using a plurality of second stimulation parameters. In the illustrated embodiment, control circuit 106B includes a signal receiver 108B, a physiologic change detector 110B, and a parameter adjuster 112B. In various embodiments, control circuit 106B can include parameter adjuster 112B and either or both of signal receiver 108B and physiologic change detector 110B.

In one embodiment, signal receiver 108B receives a second signal indicative of delivery of the first type electrical stimulation, such as from stimulation circuit 102A. Parameter adjuster 112B adjusts at least one parameter of the plurality of second stimulation parameters in response to a receipt of the second signal. In another embodiment, physiologic change detector 110B detects a second type physiologic change indicative of delivery of the first type electrical stimulation. Parameter adjuster 112B adjusts at least one parameter of the plurality of second stimulation parameters in response to a detection of the second type physiologic change. In another embodiment, signal receiver 108B receives a second signal indicative of delivery of the first type electrical stimulation, such as from stimulation circuit 102B. Physiologic change detector 110B starts a second detection window in response to the receipt of the second signal, and detects the second type physiologic change during the second detection window. Parameter adjuster 112B adjusts at least one parameter of the plurality of second stimulation parameters in response to a detection of the second type physiologic change during the second detection window.

In various embodiments, to detect the second type physiologic change, physiologic change detector 110B receives a physiologic signal, produces a physiologic parameter using the physiologic signal, and compares the physiologic parameter to a threshold. In one embodiment, the threshold is constant over time. In another embodiment, the threshold is adjusted over time by, for example, averaging values of the physiologic parameter measured using the physiologic signal over a predetermined time period. Parameter adjuster 112B adjusts at least one parameter of the plurality of second stimulation parameters in response to the physiologic parameter exceeding the threshold. Examples of the second type physiologic change include changes in cardiac conductions as an expected response to the delivery of the second type electrical stimulation. More specific examples of the first type physiologic change include changes in cardiac conduction intervals, such as a change in AV interval or PR interval. In some embodiments, the first and second type physiologic changes includes one or more common types of physiologic changes, such as when the first and second type electrical stimulation can modulate the same one or more cardiac conditions.

In one embodiment, an implantable medical device (IMD) includes both stimulation circuits 102A and 102B, with a controller configured to function as both control circuits 106A and 106B. In other words, stimulation circuits 102A and 102B are contained in a single IMD housing. In another embodiment, a first implantable medical device includes stimulation circuit 102A, and second IMD includes stimulation circuit 102B. In other words, stimulation circuits 102A and 102B are contained in different IMD housings. In various embodiments, stimulation circuits 102A and 102B are communicatively coupled to each other through a wired or wireless communication link 114, which allows for transmission of signals between stimulation circuits 102A and 102B, such as the first and second signals. In various embodiments, detections of the first and/or second type physiologic change may be communicated between stimulation circuits 102A and 102B. If the first and second type physiologic changes include a common type physiologic change, this eliminates the need to redundant detection by both circuits.

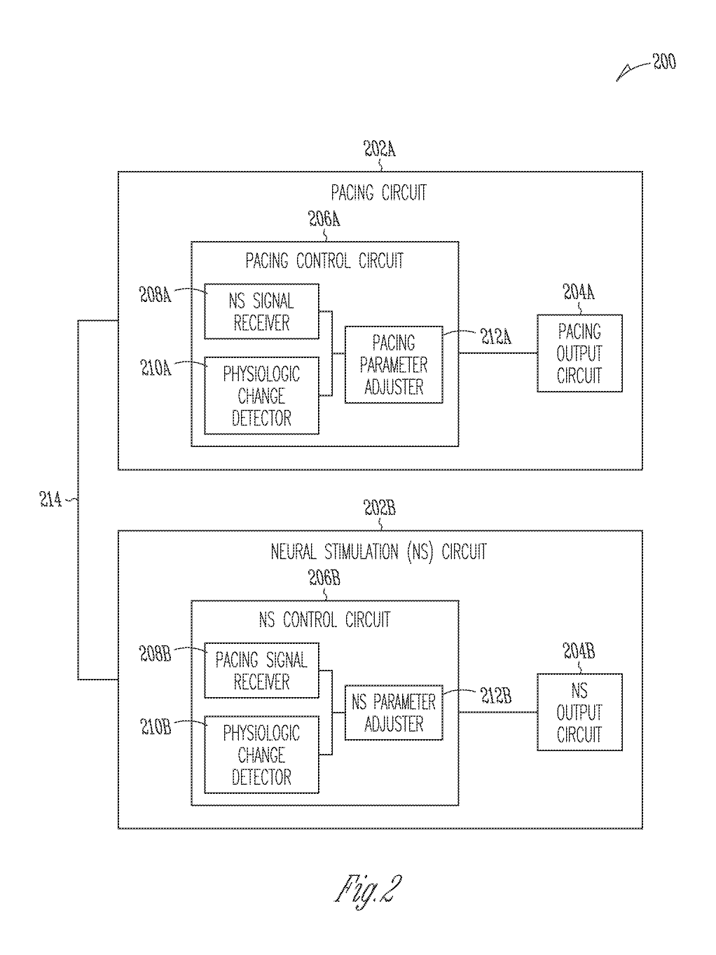

FIG. 2 is a block diagram illustrating an embodiment of a system 200 including a pacing circuit 202A and an NS circuit 202B. System 200 is an example of system 100, with pacing circuit 202A being an example of stimulation circuit 102A and NS circuit 202B being an example of stimulation circuit 102B.

Pacing circuit 202A delivers cardiac pacing to the patient's heart and includes a pacing output circuit 204A and a pacing control circuit 206A. Pacing output circuit 204A is an example of stimulation output circuit 104A and delivers pacing pulses. Pacing control circuit 206A is an example of control circuit 106A and controls the delivery of the pacing pulses using a plurality of pacing parameters. In the illustrated embodiment, pacing control circuit 206A includes an NS signal receiver 208A, a physiologic change detector 210A, and a pacing parameter adjuster 212A, which are examples of signal receiver 108A, physiologic change detector 110A, and parameter adjuster 112A, respectively. In various embodiments, pacing control circuit 206A can include pacing parameter adjuster 212A and either or both of NS signal receiver 208A and physiologic change detector 210A.

In one embodiment. NS signal receiver 208A receives an NS signal indicative of delivery of NS, such as from NS circuit 202B. Pacing parameter adjuster 212A adjusts at least one parameter of the plurality of pacing parameters in response to a receipt of the NS signal. In another embodiment, physiologic change detector 210A detects a first type physiologic change indicative of delivery of the NS. Pacing parameter adjuster 212A adjusts at least one parameter of the plurality of pacing parameters in response to a detection of the first type physiologic change. In another embodiment, NS signal receiver 208A receives the NS signal indicative of delivery of the NS, such as from NS circuit 202B. Physiologic change detector 210A starts a first detection window in response to the receipt of the NS signal, and detects the first type physiologic change during the first detection window. Pacing parameter adjuster 212A adjusts at least one parameter of the plurality of pacing parameters in response to a detection of the first type physiologic change during the first detection window.

In various embodiments, to detect the first type physiologic change, physiologic change detector 210A receives a physiologic signal, produces a physiologic parameter using the physiologic signal, and compares the physiologic parameter to a threshold. Pacing parameter adjuster 212A adjusts at least one parameter of the plurality of pacing parameters in response to the physiologic parameter exceeding the threshold. Examples of the first type physiologic change include changes in cardiac conductions as an expected response to the delivery of NS from NS circuit 202B. More specific examples of the first type physiologic change include changes in cardiac conduction intervals, such as a change in the AV interval or PR interval. In one embodiment, to detect the change in AV interval, physiologic change detector 210A receives an intracardiac electrogram, measures the AV interval from the electrogram, and compares the measured AV interval to a threshold AV interval. In various embodiments, examples of the pacing parameters that are adjustable by pacing parameter adjuster 212A include AV timing parameter (such as AV delay), interventricular or VV timing parameter (such as interventricular or VV delay), pacing site, pacing chamber, pacing amplitude, pacing pulse width, pacing energy, pacing mode, refractory interval, sensing threshold, pacing rate limiter (such as lower rate limit, maximum tracking rate, and maximum sensor rate), and adaptive rate parameter.

NS circuit 202B delivers NS to the patient's nervous system and includes an NS output circuit 204B and an NS control circuit 206B. NS output circuit 204B is an example of stimulation output circuit 104B and delivers NS pulses. In another example, NS output circuit 204B delivers non-pulsed (e.g., continuous) energy. NS control circuit 206B is an example of control circuit 106B and controls the delivery of the NS pulses using a plurality of NS parameters. In the illustrated embodiment, NS control circuit 206B includes a pacing signal receiver 208B, a physiologic change detector 210B, and an NS parameter adjuster 212B, which are examples of signal receiver 108B, physiologic change detector 110B, and parameter adjuster 112B, respectively. In various embodiments, NS control circuit 206B can include NS parameter adjuster 212B and either or both of pacing signal receiver 208B and physiologic change detector 210B.

In one embodiment, pacing signal receiver 208B receives a pacing signal indicative of delivery of pacing pulses, such as from pacing circuit 202A. NS parameter adjuster 212B adjusts at least one parameter of the plurality of NS parameters in response to a receipt of the pacing signal. In another embodiment, physiologic change detector 210B detects a first type physiologic change indicative of delivery of the cardiac pacing. NS parameter adjuster 212B adjusts at least one parameter of the plurality of NS parameters in response to a detection of the first type physiologic change. In another embodiment, pacing signal receiver 208B receives the pacing signal indicative of delivery of the cardiac pacing, such as from pacing circuit 202A. Physiologic change detector 210B starts a second detection window in response to the receipt of the pacing signal, and detects a second type physiologic change during the second detection window. NS parameter adjuster 212B adjusts at least one parameter of the plurality of NS parameters in response to a detection of the second type physiologic change during the second detection window.

In various embodiments, to detect the second type physiologic change, physiologic change detector 210B receives a physiologic signal, produces a physiologic parameter using the physiologic signal, and compares the physiologic parameter to a threshold. NS parameter adjuster 212B adjusts at least one parameter of the plurality of NS parameters in response to the physiologic parameter exceeding the threshold. Examples of the second type physiologic change include changes in cardiac conductions as an expected response to the delivery of cardiac pacing from pacing circuit 202A. More specific examples of the first type physiologic change include changes in cardiac conduction intervals, such as a change in the AV interval or PR interval. In some embodiments, the first and second type physiologic changes includes one or more common types of physiologic changes, such as when the first and second type electrical stimulation can modulate the same one or more cardiac conditions. In various embodiments, examples of the NS parameters that are adjustable by NS parameter adjuster 212B include NS timing parameter, stimulation site, NS energy parameter, and parameter temporally coordinating the NS to the cardiac pacing.

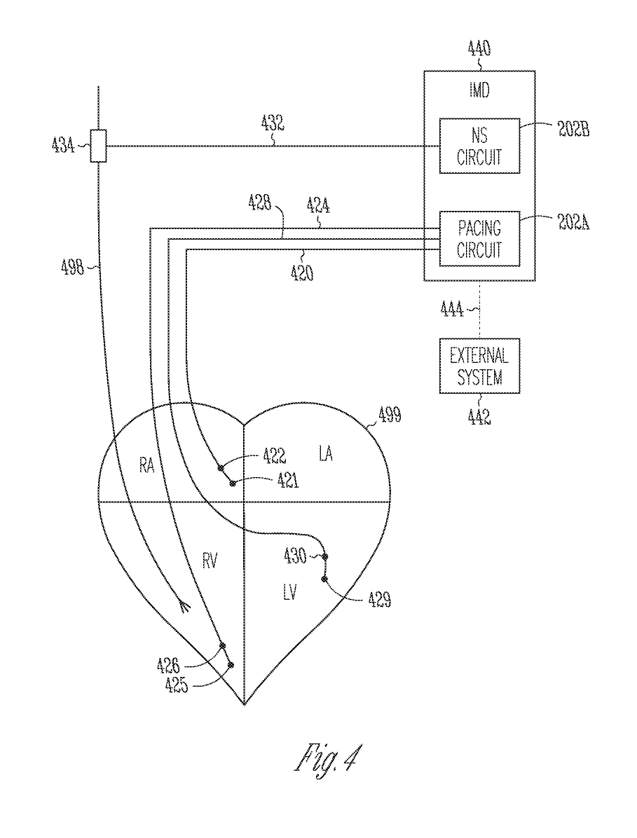

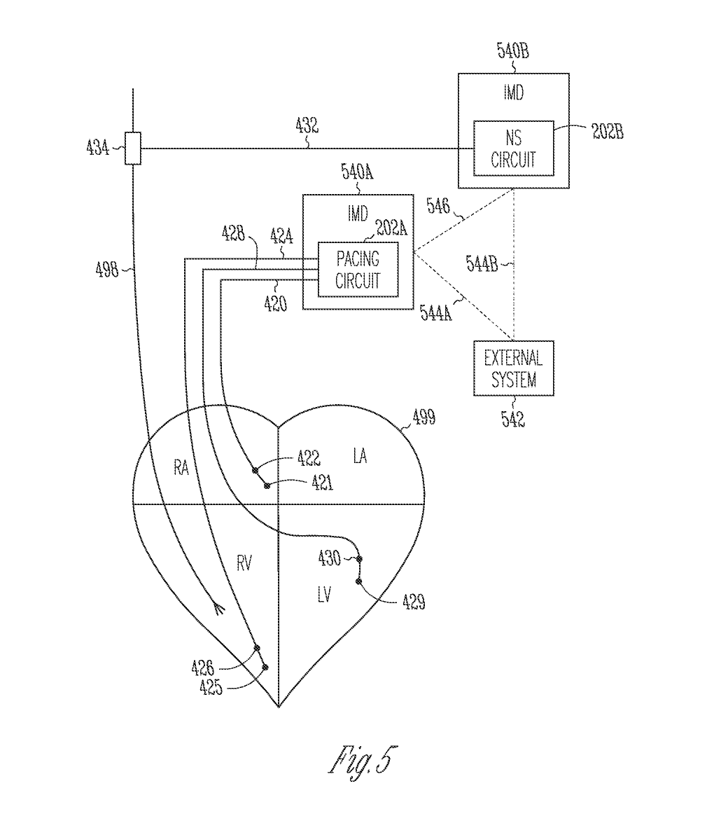

In various embodiments, pacing circuit 202A and NS circuit 202B may be included in one or more IMDs, as further discussed below with reference to FIGS. 4 and 5. In various embodiments, pacing circuit 202A and NS circuit 202B are communicatively coupled to each other through a wired or wireless communication link 214, which allows for transmission of signals between stimulation circuits pacing circuit 202A and NS circuit 202B, such as the pacing and NS signals. In various embodiments, detections of the first and/or second type physiologic change may be communicated between pacing circuit 202A and NS circuit 202B. If the first and second type physiologic changes include a common type physiologic change, this eliminates the need to redundant detection by both circuits. For example, if the common type physiologic change is a change in the AV interval, physiologic change detector 210A detects the AV interval and communicates the detection to NS control circuit 206B if needed.

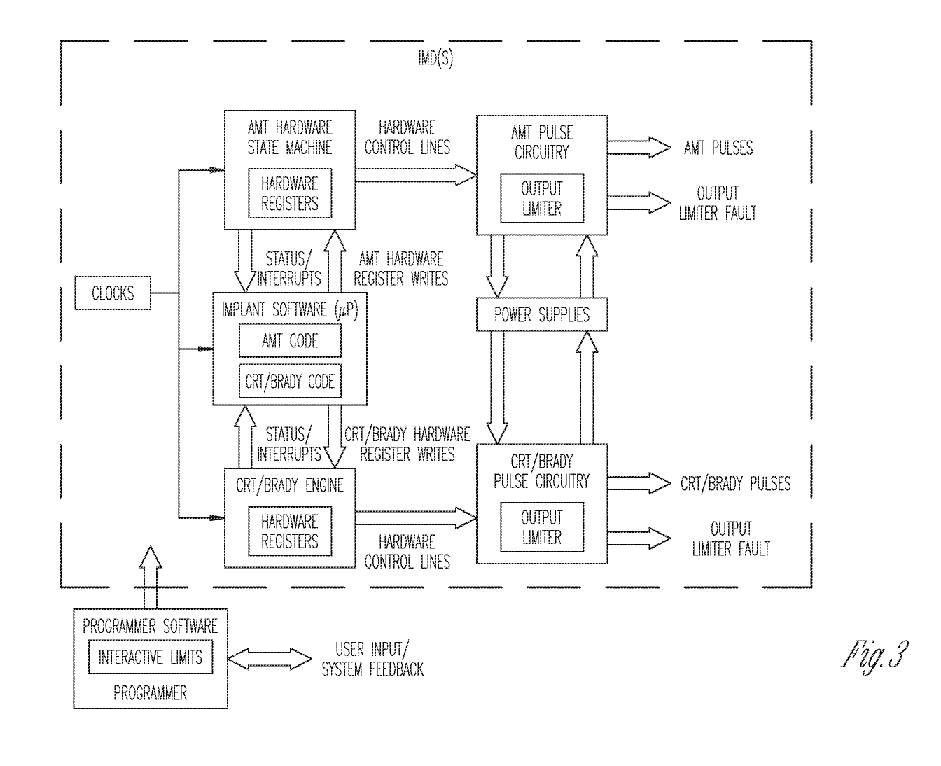

FIG. 3 is a block diagram illustrating an embodiment of a circuit for system 200 as implemented in one or more IMDs. For the purpose of illustration, the circuit is configured to deliver AMT as an example of the NS, and cardiac resynchronization therapy (CRT) and/or bradycardia pacing therapy (Brady) as examples of the cardiac pacing. The circuit is implemented using a combination of hardware and software, with pacing control circuit 206A and NS control circuit 206B implemented in a microprocessor (μP) based control circuit that includes AMT hardware state machine (including hardware registers), CRT/Brady engine (including hardware registers), and implant software including AMT code and CRT/Brady code executed by the microprocessor. Pacing output circuit 204A is implemented in CRT/Brady pulse circuitry configured for delivering pacing pulses and including output limiters that prevents potentially harmful faulty signals from being delivered to the patient's heart. NS output circuit 204B is implemented in AMT pulse circuitry configured for delivering NS pulses and including output limiters that prevents potentially harmful faulty signals from being delivered to the patient's autonomic nervous system. In various embodiments, a programmer communicatively coupled to the one or more IMDs to allow for control of the operation of one or more IMDs by a user such as a physician or other caregiver.

In various embodiments, the circuit for system 100 or 200 may be implemented using a combination of hardware and software. In various embodiments, each element of stimulation circuits 102A and 102B, including their various embodiments, may be implemented using an application-specific circuit constructed to perform one or more particular functions or a general-purpose circuit programmed to perform such function(s). Such a general-purpose circuit includes, but is not limited to, a microprocessor or portions thereof, a microcontroller or portions thereof, and a programmable logic circuit or portions thereof.

FIG. 4 is an illustration of an embodiment of an implantable system for modulating cardiovascular functions using pacing circuit 202A and NS circuit 202B and portions of the patient's body in which the implantable system is implanted to operate. The implantable system includes an IMD 440 that is electrically coupled to the patient's heart 499 through implantable leads 420, 424, and 428 and a nerve 498 (such as a branch of the autonomic nervous system) of the patient through an implantable lead 432. An external system 442 communicates with IMD 440 via a telemetry link 444.

IMD 440 includes a hermetically sealed can housing portions of pacing circuit 202A and NS circuit 202B. In various embodiments, the hermetically sealed can also functions as an electrode (referred to as “can electrode” hereinafter) for sensing and/or pulse delivery purposes. In various embodiments, IMD 440 may also include one or more devices selected from monitoring devices and therapeutic devices such as cardioverter/defibrillator, drug delivery device, and biological therapy device.

Lead 420 is a right atrial (RA) pacing lead that includes an elongate lead body having a proximal end configured to be connected to pacing circuit 202A and a distal end configured for placement in the RA in or near the atrial septum (RA). Lead 420 includes an RA tip electrode 421 and an RA ring electrode 422. RA electrodes 421 and 422 are incorporated into the lead body at the distal end for placement in or near the atrial septum, and can each be electrically connected to IMD 440 through a conductor extending within the lead body. RA tip electrode 421. RA ring electrode 422, and/or the can electrode allow for sensing an RA electrogram indicative of RA depolarizations and delivering RA pacing pulses.

Lead 424 is a right ventricular (RV) pacing lead that includes an elongate lead body having a proximal end configured to be connected to pacing circuit 202A and a distal end configured for placement in the right ventricle (RV). Lead 424 includes an RV tip electrode 425 and an RV ring electrode 426. RV electrodes 425 and 426 are incorporated into the lead body at the distal end for placement in the RV at or near the RV apex. Electrodes 425 and 426 can each be electrically connected to IMD 440 through a conductor extending within the lead body. RV tip electrode 425, RV ring electrode 426, and/or the can electrode allow for sensing an RV electrogram indicative of RV depolarizations and delivering RV pacing pulses.

Lead 428 is a left ventricular (LV) coronary pacing lead that includes an elongate lead body having a proximal end configured to be connected to pacing circuit 202A and distal end configured for placement in the coronary vein over the left ventricle (LV). Lead 125 includes an LV tip electrode 429 and an LV ring electrode 430. The distal portion of lead 428 is configured for placement in the coronary sinus and coronary vein such that LV electrodes 429 and 430 are placed in the coronary vein. LV electrodes 429 and 430 are incorporated into the lead body at the distal end and can each be electrically connected to IMD 440 through a conductor extending within the lead body. LV tip electrode 429 and LV ring electrode 430, and/or the can electrode allow for sensing an LV electrogram indicative of LV depolarizations and delivering LV pacing pulses.

In various embodiments, leads 420, 424, and/or 428 may also include defibrillation electrodes allowing for delivery of cardioversion/defibrillation pulses to heart 499. Electrodes from different leads may also be used to sense an electrogram or deliver pacing or cardioversion/defibrillation pulses.

Lead 432 is an NS lead that includes an elongate lead body having a proximal end configured to be connected to NS circuit 202B and a distal end configured for placement on or near nerve 498. Lead 432 includes an NS electrode 434, which is incorporated into the lead body at the distal end for placement on or near nerve 434, and can be electrically connected to IMD 440 through one or more conductors extending within the lead body. In various embodiments, NS electrode 434 represents an electrode or electrode array allowing for sensing one or more neural signals and delivering NS pulses. Examples of NS electrode 434 include unipolar, bipolar, or multipolar nerve cuff electrodes.

The lead configuration including RA lead 420, RV lead 424, LV lead 428, and NS lead 432 is illustrated in FIG. 4 by way of example and not by way of restriction. Other lead configurations may be used, depending on monitoring and therapeutic requirements. For example, additional one or more leads may be used to provide access to additional cardiac regions in heart 499, and leads 420, 424, and 428 may each include more or fewer electrodes along the lead body at, near, and/or distant from the distal end, depending on specified cardiac monitoring and therapeutic needs. Likewise, additional one or more leads may be used to provide access to additional nerve(s) or nerve branch(es) in the patient's body, and each lead may include any number of electrodes distributed along the lead, depending on specified neural monitoring and therapeutic needs.

External system 442 allows for programming of IMD 440 and receives signals acquired by IMD 440. In one embodiment, external system 440 includes a programmer. In another embodiment, external system 440 includes a patient monitoring system. In one embodiment, telemetry link 444 includes an inductive telemetry link. In another embodiment, telemetry link 444 includes a far-field radio-frequency telemetry link. Telemetry link 444 provides for data transmission from IMD 440 to external system 442. This may include, for example, transmitting real-time physiologic data acquired by IMD 440, extracting physiologic data acquired by and stored in IMD 440, extracting therapy history data stored in IMD 440, and extracting data indicating an operational status of IMD 440 (e.g., battery status and lead impedance). Telemetry link 440 also provides for data transmission from external system 442 to IMD 440. This may include, for example, programming IMD 440 to acquire physiologic data, programming IMD 440 to perform at least one self-diagnostic test (such as for a device operational status), programming IMD 440 to run a signal analysis algorithm, and programming IMD 440 to deliver pacing and NS therapies, including programming various rules controlling coordination between the pacing and NS therapies.

FIG. 5 is an illustration of another embodiment of an implantable system for modulating cardiovascular functions using pacing circuit 202A and NS circuit 202B and portions of a patient's body in which the implantable system is implanted to operate. The implantable system illustrated in FIG. 5 differs from the implantable system illustrated in FIG. 5 in that an IMD 540A includes pacing circuit 202A and an IMD 540B includes NS circuit 202B. An external system 542 communicates with IMD 540A via a telemetry link 544A and communicates with IMD 540B via a telemetry link 544B. IMD 540A and IMD 540B communicates with each other via a telemetry link 546 (thereby allowing pacing circuit 202A and NS circuit 202B to be communicatively coupled to each other through wireless communication link 214). In various embodiments, IMD 540A and IMD 540B can be implanted in different regions of the patient's body. In various embodiments, different telemetry modes can be used for intrabody communication (e.g. conducted electrical communication mode) and extracorporeal communication (e.g. RF communication mode).

IMD 540A includes a hermetically sealed can housing portions of pacing circuit 202A. The hermetically sealed can also functions as a can electrode for sensing and/or pulse delivery purposes. In various embodiments, IMD 540A may also include one or more devices selected from monitoring devices and therapeutic devices such as cardioverter/defibrillator, drug delivery device, and biological therapy device.

IMD 540B includes a hermetically sealed can housing portions of NS circuit 202B. The hermetically sealed can also functions as another can electrode for sensing and/or pulse delivery purposes. In various embodiments, IMD 540B may also include one or more devices selected from monitoring devices and therapeutic devices such as cardioverter/defibrillator, drug delivery device, and biological therapy device.

External system 542 is substantially similar to external system 442 except for supporting two telemetry links 544A and 544B. In various embodiments, external system 542 is configured to perform the same functions of external system 442 when IMDs 540A and 540B together are configured to perform the same functions of IMD 440. In various embodiments, the lead system, including leads 420, 424, 428, and 432 as an example, can be used with either the implantable system illustrated in FIG. 4 or the implantable system illustrated in FIG. 5.

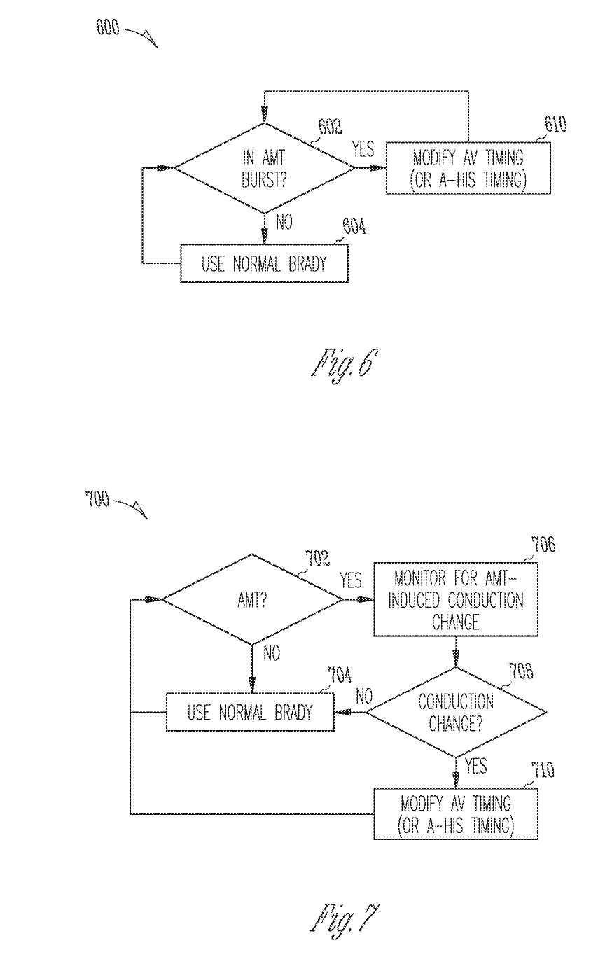

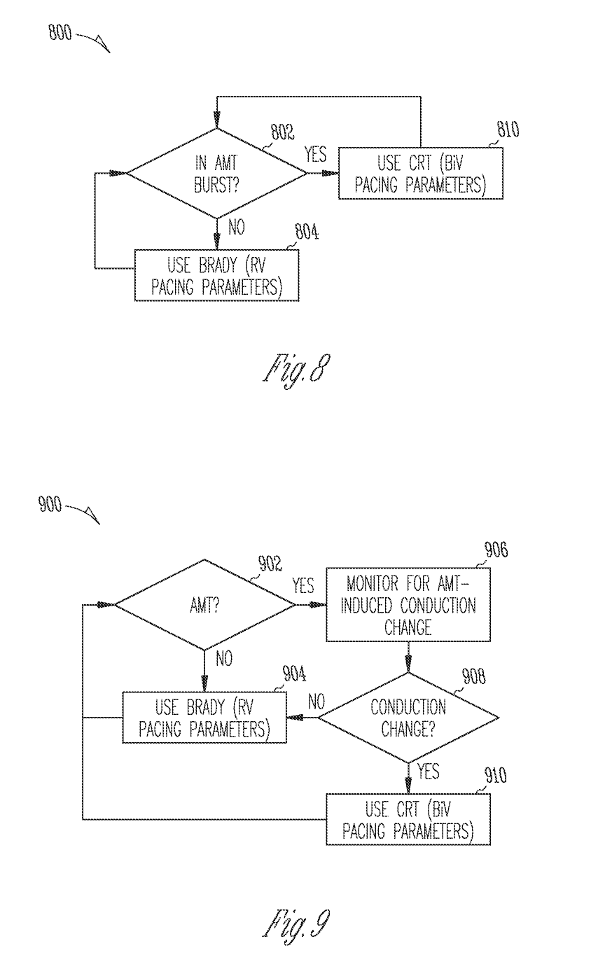

FIGS. 6-9 illustrate adjustment of cardiac pacing in response to AMT-induced changes in cardiac conduction. AMT can affect cardiac conduction, such as atrioventricular (AV) nodal conduction and bundle branch conduction, and result in change in one or more cardiac conduction intervals. Pacing parameters such as AV timing and number and/or location of ventricular pacing site(s) may be adjusted to account for the AMT-induced cardiac conduction change. Under some circumstances, delivery of AMT results in cardiac conduction dysfunction, such as AV nodal dysfunction and bundle branch dysfunction. Pacing parameters such as AV timing and number and/or location of ventricular pacing site(s) may be adjusted to mitigate such dysfunction. FIGS. 6 and 7 illustrate the adjustment of AV timing when AMT is delivered during a bradycardia pacing therapy. FIGS. 8 and 9 illustrate the adjustment of ventricular pacing site(s) when AMT is delivered, i.e., switching between bradycardia pacing therapy and CRT.

FIG. 6 is a flow chart illustrating an embodiment of a method 600 for adjusting a pacing therapy when an AMT is delivered. In one embodiment, method 600 is performed using system 200. For example, pacing control circuit 206A can be configured to perform method 600, among other things.

Method 600 is applied for delivering AMT and bradycardia pacing therapy to the patient's body. If a burst of NS pulses of the AMT is being delivered at 602, AV timing (such as AV delay) is modified for the bradycardia pacing at 610. If His-bundle pacing is delivered as the bradycardia pacing therapy, A-His timing (instead of AV timing) is modified at 610. If the NS pulses of the AMT are not being delivered at 602, a normal bradycardia pacing therapy (“NORMAL BRADY”) is delivered at 604. The “NORMAL BRADY” in FIGS. 6 and 7 refers to the bradycardia pacing therapy with pacing parameters not being adjusted to account for the AMT-induced conduction change.

FIG. 7 is a flow chart illustrating an embodiment of a method 700 for adjusting a pacing therapy when an AMT is delivered. In one embodiment, method 600 is performed using system 200. For example, pacing control circuit 206A can be configured to perform method 700, among other things.

Method 700 is applied for delivering AMT and bradycardia pacing therapy to the patient's body. If a burst of NS pulses of the AMT is being delivered at 702, AMT-induced conduction change is monitored for at 706. If the AMT-induced conduction change is detected at 708, AV timing (such as AV delay) is modified for the bradycardia pacing at 710. If His-bundle pacing is delivered as the bradycardia pacing therapy, A-His timing (instead of AV timing) is modified at 710. If the NS pulses of the AMT are not being delivered at 702, or if the AMT-induced conduction change is not detected at 708, a normal bradycardia pacing therapy (“NORMAL BRADY”) is delivered at 704.

FIG. 8 is a flow chart illustrating another embodiment of a method 800 for adjusting a pacing therapy when an AMT is delivered. In one embodiment, method 800 is performed using system 200. For example, pacing control circuit 206A can be configured to perform method 800, among other things.

Method 800 is applied for delivering AMT, bradycardia pacing therapy, and CRT to the patient's body. If a burst of NS pulses of the AMT is being delivered at 802, CRT (using biventricular or left ventricular only pacing parameters) is applied at 810. If the NS pulses of the AMT are not being delivered at 802, bradycardia pacing therapy (using right ventricular pacing parameters) is applied at 804.

FIG. 9 is a flow chart illustrating an embodiment of a method 900 for adjusting a pacing therapy when an AMT is delivered. In one embodiment, method 900 is performed using system 200. For example, pacing control circuit 206A can be configured to perform method 900, among other things.

Method 900 is applied for delivering AMT, bradycardia pacing therapy, and CRT to the patient's body. If a burst of NS pulses of the AMT is being delivered at 802, AMT-induced conduction change is monitored for at 906. If the AMT-induced conduction change (e.g., a change in QRS width or atrioventricular interval) is detected at 908, CRT (using biventricular or left ventricular only pacing parameters) is applied at 910. If the NS pulses of the AMT are not being delivered at 902, or if the AMT-induced conduction change is not detected at 908, bradycardia pacing therapy (using right ventricular pacing parameters) is applied at 904.

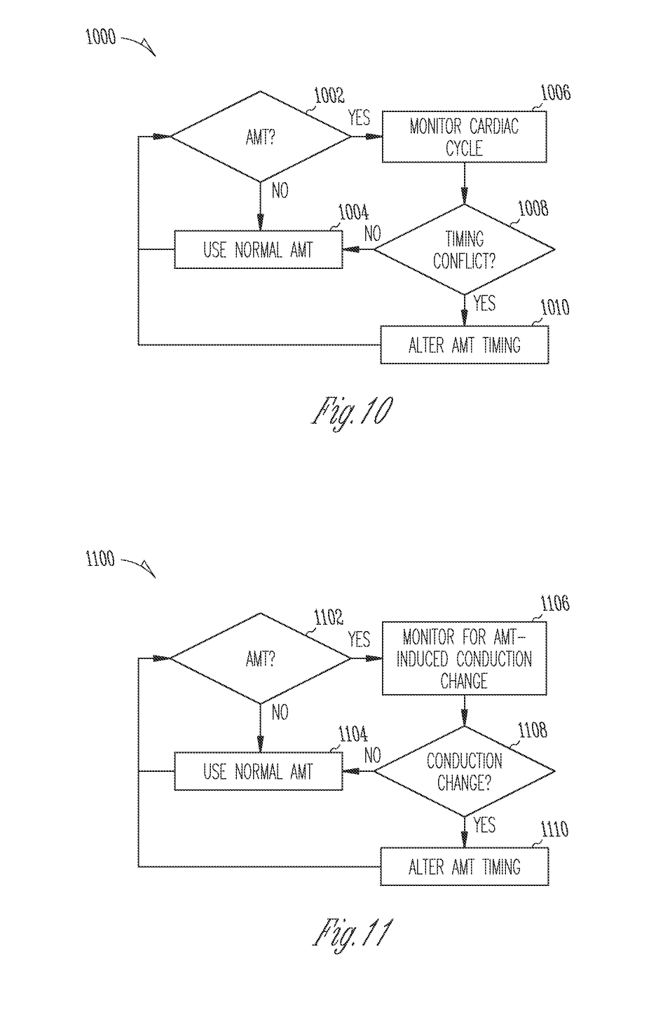

FIGS. 10 and 11 illustrate adjustment of AMT in response to AMT-induced changes in cardiac conduction. AMT can affect cardiac conduction, such as atrioventricular (AV) nodal conduction and bundle branch conduction, and result in change in one or more cardiac conduction intervals. NS parameters such as AMT timing relative to the patient's cardiac cycles may be adjusted to account for the AMT-induced cardiac conduction change. Under some circumstances, delivery of AMT results in cardiac conduction dysfunction, such as AV nodal dysfunction and bundle branch dysfunction. NS parameters such as AMT timing relative to the patient's cardiac cycles may be adjusted to mitigate such dysfunction. In various embodiments, AMT include vagus nerve stimulation, spinal cord stimulation, baroreceptor stimulation, and/or other stimulation therapy intended to modulate neural activities in the autonomic nervous system.

FIG. 10 is a flow chart illustrating an embodiment of a method 1000 for adjusting an AMT therapy. In one embodiment, method 1000 is performed using system 200. For example, NS control circuit 206B can be configured to perform method 1000, among other things.

Method 1000 is applied for delivering AMT to the patient's body. If the AMT is being delivered at 1002, the patient's cardiac cycle is monitored for its timing relative to the NS pulses of the AMT. If a timing conflict is detected at 1008, the timing of the AMT is altered at 1010. In various embodiments, the timing conflict is detected if a burst of NS pulses of the AMT is delivered during a period of the cardiac cycle when an unintended or undesirable change in cardiac conduction is expected to be induced by the AMT. The timing of delivery of the burst of NS pulses of the AMT is altered at 1010 such that it falls into a period of cardiac cycle during which the unintended or undesirable conduction change is not expected to be induced by the AMT. If the AMT is not delivered at 1002, or if the AMT-induced conduction change is not detected at 1108, the normal AMT is applied at 1004. The “NORMAL AMT” in FIGS. 10 and 11 refers to the AMT with NS parameters not being adjusted to avoid the timing conflict or AMT-induced condition change.

FIG. 11 is a flow chart illustrating an embodiment of a method 1100 for adjusting an AMT therapy. In one embodiment, method 1100 is performed using system 200. For example, NS control circuit 206B can be configured to perform method 1100, among other things.

Method 1100 is applied for delivering AMT to the patient's body. If the AMT is being delivered at 1002, AMT-induced conduction change is monitored for at 1106. If the conduction change is detected at 1108, the timing of the AMT is altered at 1110. In various embodiments, the timing of delivery of the burst of NS pulses of the AMT may be adjusted, such as incrementally, at 1110, until the AMT-induced conduction change is no longer detected at 1108. If the AMT is not delivered at 1102, or if the AMT-induced conduction change is not detected at 1108, the normal AMT is applied at 1104.

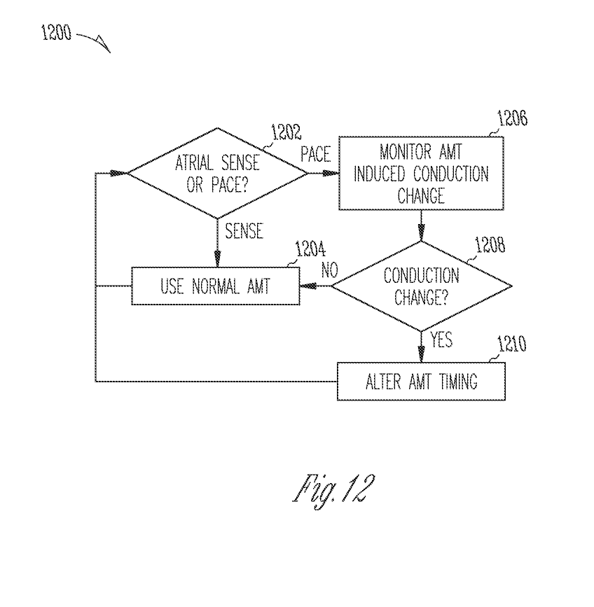

FIG. 12 illustrates adjustment of AMT in response to pacing-dependent AMT-induced changes in cardiac conduction. AMT can affect cardiac conduction, such as atrioventricular (AV) nodal conduction and bundle branch conduction, and result in change in one or more cardiac conduction intervals. Such effects of AMT may differ depending on whether the patient's atrial events are intrinsic or paced. NS parameters such as AMT timing relative to the patient's cardiac cycles may be adjusted to account for the AMT-induced cardiac conduction change. Under some circumstances, delivery of AMT results in cardiac conduction dysfunction, such as AV nodal dysfunction and bundle branch dysfunction. NS parameters such as AMT timing relative to the patient's cardiac cycles may be adjusted to mitigate such dysfunction. When the effects of AMT depend on whether the patient's atrial events are intrinsic or paced, whether to adjust the AMT timing may also depend on whether the patient's atrial events are intrinsic or paced.

FIG. 12 is a flow chart illustrating an embodiment of a method 1200 for adjusting an AMT therapy when a pacing therapy is delivered. In one embodiment, method 1200 is performed using system 200. For example, NS control circuit 206B can be configured to perform method 1200, among other things.

Method 1200 is applied for delivering AMT and cardiac pacing to the patient's body. If the cardiac pacing is delivered with an atrial event being a paced event at 1202, AMT-induced conduction change is monitored for at 1206. If the conduction change is detected at 1208, the timing of the AMT is altered at 1210. In various embodiments, the timing of delivery of the burst of NS pulses of the AMT may be adjusted, such as incrementally, at 1210, until the AMT-induced conduction change is no longer detected at 1208. If the cardiac pacing is delivered with the atrial event being a sensed (intrinsic) event at 1202, or if the AMT-induced conduction change is not detected at 1208, the normal AMT is applied at 1204. The “NORMAL AMT” in FIG. 12 refers to the AMT with NS parameters not being adjusted to avoid the pacing-dependent AMT-induced conduction change.

In various embodiments, a detection window (time window) is applied for monitoring for the AMT-induced conduction change (e.g., at 706, 906, 1106, and 1206). The AMT-induced conduction change is to be detected during the detection window. The detection window can be a function of when the burst of AMT pulses is delivered relative to the cardiac cycle of the patient. For example, AMT may only affect AV timing when an AMT pulse is delivered at certain period during a cardiac cycle (such as within 400 to 500 milliseconds after a P-wave). Thus, in one embodiment, the detection window is applied only during the time periods when the delivery of AMT is expected to affect the AV timing and require modification of the AV timing pacing parameter.

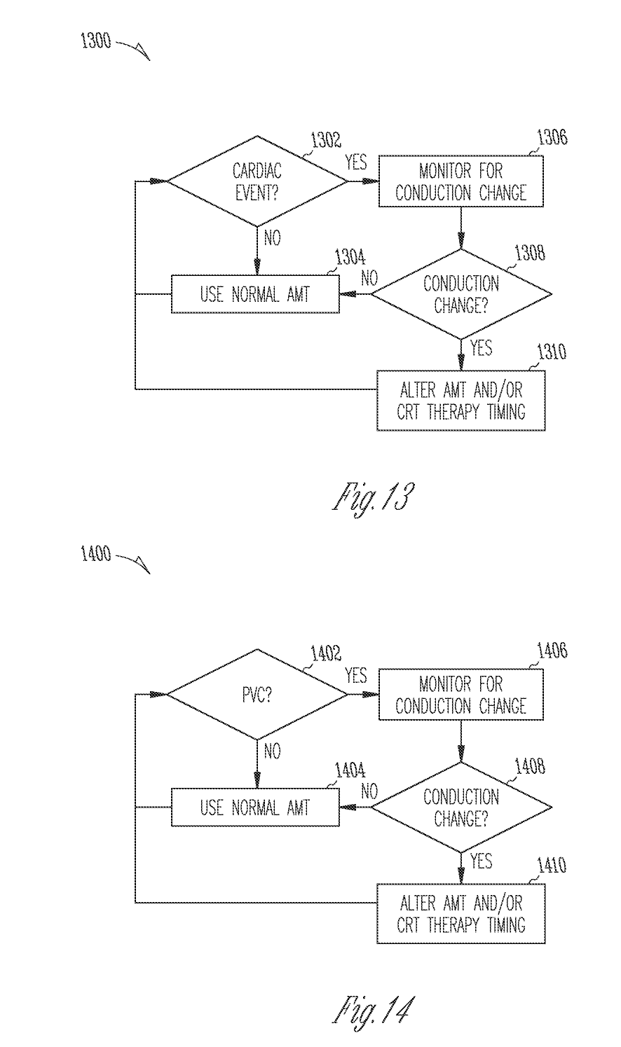

FIGS. 13 and 14 illustrate adjustment of AMT in response to cardiac event-dependent AMT-induced changes in cardiac conduction. AMT can affect cardiac conduction, such as atrioventricular (AV) nodal conduction and bundle branch conduction, and result in change in one or more cardiac conduction intervals. Such effects of AMT may differ depending on the patient's various cardiac events that occur during the delivery of the AMT. NS parameters such as AMT timing relative to the patient's cardiac cycles may be adjusted to account for the AMT-induced cardiac conduction change. If a pacing therapy such as CRT is delivered, pacing parameters controlling timing of the CRT may also be adjusted to account for the AMT-induced cardiac conduction change. Under some circumstances, delivery of AMT results in cardiac conduction dysfunction, such as AV nodal dysfunction and bundle branch dysfunction. NS parameters such as AMT timing relative to the patient's cardiac cycles and/or pacing parameters may be adjusted to mitigate such dysfunction. When the effects of AMT depend on particular cardiac events occurring in the patient, the adjustment of NS and/or pacing parameters may also depend on the type(s) of the cardiac events detected.

FIG. 13 is a flow chart illustrating an embodiment of a method 1300 for adjusting an AMT therapy when a cardiac event is detected. In one embodiment, method 1300 is performed using system 200. For example, NS control circuit 206B can be configured to perform method 1300, among other things.

Method 1300 is applied for delivering AMT and cardiac pacing such as CRT to the patient's body. If a cardiac event of one of specified types is detected at 1302, AMT-induced conduction change is monitored for at 1306. If the conduction change is detected at 1308, the timing of the AMT and/or CRT is altered at 1310. In various embodiments, the timing of delivery of the burst of NS pulses of the AMT may be adjusted, and/or the timing of delivery of the pacing pulses of the CRT may be adjusted, such as incrementally, at 1310, until the AMT-induced conduction change is no longer detected at 1308. If the cardiac event of the one of the specified types is not detected at 1302, or if the AMT-induced conduction change is not detected at 1308, the normal AMT is applied at 1304. The “NORMAL AMT” in FIGS. 13 and 14 refers to the AMT with NS parameters not being adjusted to avoid the cardiac event-dependent AMT-induced conduction change.

FIG. 14 is a flow chart illustrating an embodiment of a method 1400 for adjusting an AMT therapy when a premature ventricular contraction (PVC) is detected. In one embodiment, method 1400 is performed using system 200. For example, NS control circuit 206B can be configured to perform method 1400, among other things.

Method 1400 is applied for delivering AMT and cardiac pacing such as CRT, and represents an example of method 1300 with the cardiac event being a PVC. If a PVC is detected at 1402, AMT-induced conduction change is monitored for at 1406. If the conduction change is detected at 1408, the timing of the AMT and/or the CRT is altered at 1410. In various embodiments, the timing of delivery of the burst of NS pulses of the AMT may be adjusted, such as incrementally, at 1410, until the AMT-induced conduction change is no longer detected at 1408. If the PVC is not detected 1402, or if the AMT-induced conduction change is not detected at 1408, the normal AMT is applied at 1404.

In various embodiments, each of methods 600, 700, 800, 900, 1000, 1100, 1200, 1300, and 1400 can include a “learning” algorithm for determining approximately optimal timing for delivering the AMT and/or pacing therapy based on the degree of the AMT-induced conduction change in each individual patient. For example, such optimal timing may be learned acutely and applied chronically. Changes in cardiac conduction caused by different reasons (e.g., AMT, pacing, and cardiac events) may be associated with unique optimal AMT timing parameters, which in various embodiments can be determined empirically.

It is to be understood that the above detailed description is intended to be illustrative, and not restrictive. Other embodiments will be apparent to those of skill in the art upon reading and understanding the above description. The scope of the invention should, therefore, be determined with reference to the appended claims, along with the full scope of equivalents to which such claims are entitled.

What is claimed is:

1. A system for delivering a plurality of electrical stimulation therapies to a body, the system comprising:
a first stimulation circuit configured to deliver a first type electrical stimulation to the body, the first stimulation circuit including:
a first pulse output circuit configured to deliver first stimulation pulses; and
a first control circuit configured to control the delivery of the first stimulation pulses using a plurality of first stimulation parameters, the first control circuit including:
a first signal receiver configured to receive a first signal indicative of delivery of a second type electrical stimulation;
a first physiologic change detector configured to start a first detection window in response to receipt of the first signal and detect a first type physiologic change during the first detection window; and
a first parameter adjuster configured to adjust at least one parameter of the plurality of first stimulation parameters in response to a detection of the first type physiologic change.

2. The system of claim 1, wherein the first stimulation circuit comprises a cardiac pacing circuit configured to deliver cardiac pacing to the body, the first pulse output circuit comprises a pacing output circuit configured to deliver pacing pulses, and the first control circuit comprises a pacing control circuit configured to control the delivery of the pacing pulses using a plurality of pacing parameters.

3. The system of claim 2, wherein the first signal receiver comprises a neural stimulation (NS) signal receiver configured to receive an NS signal indicative of delivery of NS, the first physiologic change detector is configured to start the first detection window in response to the a receipt of the NS signal and detect a change in a cardiac conduction interval during the first detection window, and the first parameter adjuster comprises a pacing parameter adjuster configured to adjust at least one parameter of the plurality of pacing parameters in response to a detection of the change in the cardiac conduction interval.

4. The system of claim 3, wherein the first physiologic change detector is configured to detect a change in an atrioventricular interval, and the pacing parameter adjuster is configured to adjust an atrioventricular delay of the plurality of pacing parameters in response to a detection of the change in the atrioventricular interval.

5. The system of claim 1, wherein the first stimulation circuit comprises a neural stimulation (NS) circuit configured to deliver NS to the body, the first pulse output circuit comprises an NS output circuit configured to deliver NS pulses, and the first control circuit comprises an NS control circuit configured to control the delivery of the NS pulses using a plurality of NS parameters.

6. The system of claim 5, wherein the first signal receiver comprises a cardiac pacing signal receiver to receive a cardiac pacing signal indicative of delivery of a cardiac pacing therapy, the first physiologic change detector is configured to start the first detection window in response to receipt of the cardiac pacing signal and detect a change in a cardiac conduction interval during the first detection window, and the first parameter adjuster comprises an NS parameter adjuster configured to adjust at least one parameter of the plurality of NS parameters in response to a detection of the change in the cardiac conduction interval.

7. The system of claim 1, further comprising a second stimulation circuit configured to deliver the second type electrical stimulation to the body, the second stimulation circuit including:
a second pulse output circuit configured to deliver second stimulation pulses; and
a second control circuit configured to control the delivery of the second stimulation pulses using a plurality of second stimulation parameters, the second control circuit including:
a second signal receiver configured to receive a second signal indicative of delivery of the first type electrical stimulation;
a second physiologic change detector configured to start a second detection window in response to receipt of the second signal and detect a second type physiologic change during the second detection window; and
a second parameter adjuster configured to adjust at least one parameter of the plurality of second stimulation parameters in response to a detection of the second type physiologic change.

8. The system of claim 7, wherein the first type electrical stimulation comprises cardiac pacing, and the second type electrical stimulation comprises neural stimulation (NS).

9. The system of claim 8, comprising an implantable medical device including the first stimulation circuit and the second stimulation circuit.

10. The system of claim 8, comprising:
a first implantable medical device including the first stimulation circuit; and
a second implantable medical device including the second stimulation circuit.

11. A method for delivering a plurality of electrical stimulation therapies to a body, the method comprising:
delivering first stimulation pulses of a first type electrical stimulation;
controlling the delivery of the first stimulation pulses using a plurality of first stimulation parameters of the first type electrical stimulation, the controlling including:
receiving a first signal indicative of delivery of a second type electrical stimulation;
starting a first detection window in response to the first signal being received;
detecting a first type physiologic change during the first detection window; and
adjusting at least one parameter of the plurality of first stimulation parameters in response to the first type physiologic change being detected.

12. The method of claim 11, further comprising:
delivering second stimulation pulses of the second type electrical stimulation;
controlling the delivery of the second stimulation pulses using a plurality of second stimulation parameters of the second type electrical stimulation, the controlling including:
receiving a second signal indicative of delivery of the first stimulation pulses of the first type electrical stimulation;
starting a second detection window in response to the second signal being received;
detecting a second type physiologic change during the second detection window; and
adjusting at least one parameter of the plurality of first stimulation parameters in response to the second type physiologic change being detected.

13. The method of claim 12, wherein the first type electrical stimulation comprises cardiac pacing, and the second type electrical stimulation comprises neural stimulation.

14. The method of claim 13, wherein the first type physiologic change comprises a change in a cardiac conduction interval.

15. The method of claim 14, wherein the second type physiologic change comprises a change in the cardiac conduction interval.

16. The method of claim 12, wherein the first type electrical stimulation comprises cardiac pacing, delivering the first stimulation pulses comprises delivering pacing pulses, and controlling the delivery of the first stimulation pulses comprises controlling the delivery of the pacing pulses using a plurality of pacing parameters.

17. The method of claim 16, wherein detecting the first type physiologic change during the first detection window comprises detecting a change in a cardiac conduction interval, and adjusting the at least one parameter comprises adjusting at least one parameter of the plurality of pacing parameters in response to the change in the cardiac conduction interval being detected.

18. The method of claim 16, wherein the second type electrical stimulation comprises neural stimulation (NS), delivering the second stimulation pulses comprises delivering NS pulses, and controlling the delivery of the second stimulation pulses comprises controlling the delivery of the NS pulses using a plurality of NS parameters.

19. The method of claim 18, wherein detecting the second type physiologic change during the first detection window comprises detecting a change in the cardiac conduction interval, and adjusting the at least one parameter comprises adjusting at least one parameter of the plurality of NS parameters in response to the change in the cardiac conduction interval being detected.

20. The method of claim 18, comprising selecting the plurality of pacing parameters for a bradycardia pacing therapy or a cardiac resynchronization therapy, and selecting the plurality of NS parameters for an autonomic modulation therapy.
